(19) United States
(12) Patent Application Publication
(10) Pub. No.: US 2026/0191827 A1
(43) Pub. Date: Jul. 9, 2026

(54) COMPOUNDS FOR USE IN THE TREATMENT OF DISEASES AND CONDITIONS ASSOCIATED WITH NEURODEGENERATIVE DYSFUNCTION

(71) Applicant: OnTrack Therapeutics Limited, Kent (GB)

(72) Inventors: Thomas PFEIFFER, London (GB); Alleyn Thomas PLOWRIGHT, Peterborough (GB); Man-Ling HO, London (GB)

(21) Appl. No.: 18/868,742

(22) PCT Filed: May 24, 2023
(86) PCT No.: PCT/GB2023/051355

§ 371 Date: Nov. 23, 2024

(30) Foreign Application Priority Data
May 24, 2022 (GB) 2207556.8
Aug. 2, 2022 (GB) 2211273.4
Feb. 14, 2023 (GB) 2302120.7

(51) Int. Cl. A61K 31/4188; A61P 25/28

(52) U.S. Cl. CPC A61K 31/4188; A61P 25/28

(57) ABSTRACT

The present invention relates to the compound 4-(6-oxo-2-(trifluoromethyl)-3,6-dihydrochromeno[7,8-d]imidazol-8-yl)benzonitrile, also known as CF₃CN, referred to herein as the compound of Formula I, for use in the treatment of diseases and conditions associated with neurodegenerative dysfunction.

### FIELD OF THE INVENTION

The present invention relates to the compound 4-(6-oxo-2-(trifluoromethyl)-3,6-dihydrochromeno [7,8-d]imidazol-8-yl)benzonitrile, also known as CF₃CN, referred to herein as the compound of Formula I, for use in the treatment of diseases and conditions associated with neurodegenerative dysfunction.

### BACKGROUND TO THE INVENTION

The compound tropoflavin, also known as 7,8-dihydroxyflavone (7,8-DHF), is a naturally occurring flavone found in Godmania aesculifolio, Tridax procumbent and primula tree leaves. It is known to act as a potent and selective agonist of tropomyosin receptor kinase B (TrkB), which is the main signaling receptor of neurotrophin brain-derived neurotrophic factor (BDNF).

Tropoflavin has been shown to have therapeutic efficacy in several animal models including depression, Alzheimer's disease, cognitive deficits in schizophrenia, Parkinson's disease, Huntington's disease, amyotrophic lateral sclerosis, traumatic brain injury, cerebral ischemia, fragile X syndrome and Rett syndrome.

A derivative of tropoflavin, 4-(6-oxo-2-(trifluoromethyl)-3,6-dihydrochromeno [7,8-d]imidazol-8-yl)benzonitrile, also known as CF₃CN, referred to herein as the compound of Formula I, has been shown to be useful in the treatment of diseases and conditions associated with neurodegenerative dysfunction.

Neurodegenerative diseases are caused by the progressive damage or death of neurons, these are nerve cells in the brain whose primary function is to assist in the memory process. The damage or death of neurons leads to a gradual deterioration of the functions controlled by the affected part of the nervous system.

Neurodegenerative diseases are a group of disorders characterized by changes in normal neuronal functioning, leading, in most cases, to neuronal death. Most of these diseases are associated with severe neuronal loss and the impact of these diseases can be catastrophic for the patient and their carers.

According to the National Institute of Neurological Disorders and Stroke (NINDS), there are more than 600 different types of neurological disorders. For many of these disorders there are no adequate treatments and/or therapies that exist. Such disorders include: Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction; normal pressure hydrocephalus; Parkinson's disease-related cognitive dysfunction; posterior cortical atrophy; primary progressive aphasia; prion disease; progressive supranuclear palsy; Rett syndrome; schizophrenia; stroke-related cognitive dysfunction; traumatic brain injury; traumatic spinal cord injury and vascular dementia.

Movement disorders are a group of neurological conditions that cause abnormal increased movements, which may be voluntary or involuntary. Movement disorders can also cause reduced or slow movements, the speed, fluency, quality and ease of body movements can also be affected.

According to the American Association of Neurological Surgeons movement disorders are a group of neurological conditions that can be caused by dysfunction of the brain, genetic conditions or metabolic disorders. Conditions commonly associated with movement disorders include: ataxia; cervical dystonia; chorea; dystonia; functional movement disorder; Huntington's disease; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

Cognitive dysfunction is a core feature of schizophrenia and other neuropsychiatric conditions, affecting up to 75% of patients. Cognitive deficits are moderate to severe across several domains, including attention, working memory, verbal learning and memory, and executive functions. As such, cognitive deficits are now considered a central feature of schizophrenia and other neuropsychiatric conditions.

A loss or a decrease in TrkB signalling has been shown to be associated with many neurodegenerative conditions and movement disorders including Alzheimer's disease, Huntington's disease, Parkinson's disease, autism, Rett syndrome, sudden infant death syndrome, retinal ganglion cell loss ageing and traumatic brain injury. As such the identification of compounds which are able to act as agonists of the TrkB receptor are of potential use as therapeutic treatments for diseases and conditions associated with neurodegenerative dysfunction.

The compound of Formula I, 4-(6-oxo-2-(trifluoromethyl)-3,6-dihydrochromeno [7,8-d]imidazol-8-yl)benzonitrile, has been shown to be useful in the treatment of diseases and conditions associated with neurodegenerative dysfunction. As such this compound may have utility in treating these diseases for which there is a high unmet need and a huge burden on the patient and their carers.

The patent application WO2011/156479 describes compounds and methods for selective activation of TrkB which may be useful in neuroprotection, however the compound of the invention is not specifically disclosed.

The patent application WO2020/033604 discloses the compound of Formula I and a method of preparation of the compound. The application additionally describes methods of preventing or treating a BDNF and TrkB related disease or condition. The compound is tested in an in vivo mouse model of Alzheimer's disease. Results suggested that repeated administration of the compound was able to prevent synaptic loss in the 5XFAD mice.

The present application provides data from an animal model to demonstrate that the compound of Formula I may be useful in the treatment of diseases and conditions associated with neurodegenerative dysfunction.

Symptoms associated with neurodegenerative dysfunction often vary according to the particular disorder, but some common signs and symptoms overlap in most disorders. Some of the most common signs of cognitive disorder include: confusion; poor motor coordination; loss of short-term or long-term memory; identity confusion; and impaired judgment.

Some neurodegenerative disorders develop in stages where the symptoms increase in severity the further the disease progresses. Diseases such as Alzheimer's disease often begins with the patient showing very minor signs of forgetfulness, which are often indistinguishable from normal memory errors. However, as the disease progresses, the affected person's memory becomes persistently impaired.

Currently, there is no cure for neurodegenerative disorders, therefore the treatment options for patients suffering from these symptoms will include drug therapies such as memantine, donepezil, rivastigmine and galantamine. In addition, occupational therapies and environmental approaches are often required.

There remains a need in the art for an effective treatment for diseases and conditions associated with neurodegenerative disorders.

### BRIEF SUMMARY OF THE DISCLOSURE

In accordance with a first aspect of the present invention there is provided a compound of Formula I for use in the treatment of neurodegenerative diseases or conditions.

Preferably, the neurodegenerative diseases or conditions is selected from the group: Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction; normal pressure hydrocephalus; Parkinson's disease-related cognitive dysfunction; posterior cortical atrophy; primary progressive aphasia; prion disease; progressive supranuclear palsy; Rett syndrome; schizophrenia; stroke-related cognitive dysfunction; traumatic brain injury; traumatic spinal cord injury and vascular dementia.

In some embodiments the neurodegenerative diseases or conditions is treatment resistant.

Preferably the compound of Formula I is administered with one or more pharmaceutically acceptable excipients.

Preferably the compound of Formula I is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

Preferably the compound of Formula I is administered as a single daily dose. Alternatively, the compound of Formula I is administered as multiple daily doses. Further still the compound of Formula I is administered two, three, four or five times per day.

Preferably each dose comprises at least 0.001 mg of the compound of Formula I. More preferably each dose comprises between about 0.001 mg and about 500 mg of the compound of Formula I. Alternatively each dose comprises between about 500 mg and about 1000 mg of the compound of Formula I.

In a further embodiment the compound of Formula I is administered with one or more additional drug products.

In accordance with a second aspect of the present invention there is provided a method of treating neurodegenerative diseases or conditions in a subject in need thereof, the method comprising administering to the subject a therapeutically effective amount of the compound of Formula I.

In a further embodiment of the invention, the compound of Formula I can be used to treat neurological dysfunction which is present as a result of a disease or condition such as a movement disorder, schizophrenia or other neuropsychiatric condition.

In human therapeutics, the physician will determine the dosage regimen that is most appropriate according to a preventive or curative treatment and according to the age, weight, stage of the disease and other factors specific to the subject to be treated. The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, or about 1000 mg, and in some embodiments from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above-described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### BRIEF SUMMARY OF THE DRAWINGS

The present invention is described with reference to the figure listed below:

FIG. 1 details the effect of test compound on scopolamine-induced cognitive deficit in mice.

FIG. 2 details the mean exploration time(s) of two identical objects in the acquisition trial of the NOR test in the acute phase.

FIG. 3 details the mean exploration time(s) of a familiar object and a novel object in the 3 min retention trial of the NOR test in the acute phase.

FIG. 4 details the effect of acute treatment of test compound and risperidone on the discrimination index (DI) in the NOR test.

FIG. 5 details the effect of acute treatment of test compound and risperidone on the total number of line crossings in the acquisition and retention trial of the NOR test.

FIG. 6 details the mean exploration time(s) of two identical objects in the acquisition trial of the NOR test in the 20-day treatment phase.

FIG. 7 details the mean exploration time(s) of a familiar object and a novel object in the 3 min retention trial of the NOR test in the 20-day treatment phase.

FIG. 8 details the effect of 20-day treatment of test compound and risperidone on the discrimination index (DI) in the NOR test.

FIG. 9 details the effect of 20-day treatment of test compound and risperidone on the total number of line crossings in the acquisition and retention trial of the NOR test.

### DEFINITIONS

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to affect such treatment for the disease. The “therapeutically effective amount” will vary depending on the compound, the disease and its severity and the age, weight, adsorption, distribution, metabolism and excretion etc., of the patient to be treated.

“Vehicle” refers to a diluent, excipient or carrier with which a compound is administered to a subject. In some embodiments, the vehicle is pharmaceutically acceptable.

“Active ingredient” or “Active pharmaceutical ingredient” or “API” refers to the compound of the invention.

“Neurodegenerative dysfunction” refers to any of the disorders listed by the National Institute of Neurological Disorders and Stroke (NINDS)—see https://www.ninds.nih.gov/health-information/disorders for more information. In particular the neurodegenerative dysfunctions of the invention include but are not limited to Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction;

normal pressure hydrocephalus; Parkinson's disease-related cognitive dysfunction; posterior cortical atrophy; primary progressive aphasia; prion disease; progressive supranuclear palsy; Rett syndrome; schizophrenia; stroke-related cognitive dysfunction; traumatic brain injury; traumatic spinal cord injury and vascular dementia.

“Treatment resistant” is defined as the failure of a disease or disorder to respond positively or significantly to treatment.

“Movement disorders” refers to a group of neurological conditions that cause abnormal increased movements, which may be voluntary or involuntary. Movement disorders can also cause reduced or slow movements, the speed, fluency, quality and ease of body movements can also be affected.

“Diseases or conditions associated with movement disorder” refers to any disease or condition that has been diagnosed as having a movement disorder component including but not limited to: ataxia; cervical dystonia; chorea; dystonia; functional movement disorder; Huntington's disease; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

“Sensorimotor deficit” is a reduction in the sensory and motor development in a subject. Motor development is the use and coordination of muscles of the trunk, arms, legs and hands and sensory development from the environment through sight, sounds, smell, taste and hearing are both either halted during the development process of a subject or decline due to a disease or condition.

“4-(6-oxo-2-(trifluoromethyl)-3,6-dihydrochromeno [7,8-d]imidazol-8-yl)benzonitrile”, also known as “CF₃CN”, referred to herein as the “compound of Formula I” has a SMILES code N #CC1=CC═C(C2=CC(C3=CC═C4C(N═C(C(F)(F)F)N4)=C3O2)=O)C═C1 and the structure defined below:

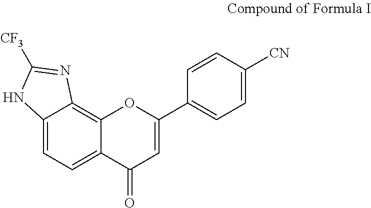 [SMILES: N#Cc1ccc(-c2cc(=O)c3ccc4[nH]c(C(F)(F)F)nc4c3o2)cc1]

### DETAILED DESCRIPTION OF THE INVENTION

The Examples below describes the effect of the compound of Formula I in animal models, which demonstrate the compound's ability to treat diseases and conditions associated with neurodegenerative dysfunction.

The scopolamine model induces a cognitive deficit in animals, the animals treated with scopolamine show a significantly impaired performance in the T-maze which is able to be reversed by the standard of care medication donepezil. In normal healthy humans, scopolamine (0.3 and 0.5 mg) reliably impaired performance on the Cognitive Drug Research test battery composite scores (power of attention, continuity of attention, quality of working memory, quality of episodic secondary memory, and speed of memory) in a dose- and time-dependent manner. Donepezil (10 mg) significantly attenuated the scopolamine-induced impairment in cognition on power of attention, continuity of attention, quality of working memory, and speed of memory.

As such reversal of scopolamine-induced cognitive impairment is a viable model for predicting pharmacodynamic signals of pro-cognitive compounds in both animals and humans.

The novel object recognition (NOR) test is a two-trial cognitive paradigm that assesses visual recognition memory. A recognition memory task allows the comparison between presented stimuli and previously stored information. Recognition memory is disturbed in a range of human disorders and NOR is widely used in rodents for investigating deficits in a variety of animal models of human conditions where cognition is impaired.

### Example 1: Effect of Test Compound on Scopolamine-Induced Cognitive Deficit in Mice

Spontaneous alternation is the innate tendency of rodents to alternate free choices in a T-maze over a series of successive runs (Dember and Fowler. 1958). This sequential procedure relies on working memory and is sensitive to various pharmacological manipulations affecting memory processes (Gerlai 1998; Stefani and Gold. 2001; Spowart-Manning and Van der Staay. 2004).

The present study was conducted to evaluate the ability of the compound of Formula I to reverse scopolamine-induced cognitive deficit in the T-maze assay.

### Materials and Methods

### Test Animals:

60 male CD-1 mice (Janvier; Le Genest St Isle—France) were used for the study, with 10 animals per group.

### Treatment:

Animals in the API groups were pre-treated (p.o.) once a day for 30 days, the last treatment was given 1 h before the T-maze test. The groups were as follows:

| Gr. # | Group label | Treatment description |
|---|---|---|
| 1 | healthy control | Saline/Vehicle |
| 2 | scopolamine-only | Scopolamine/Vehicle |
| 3 | positive control | Scopolamine/Donepezil (0.3 mg/kg) |
| 4 | scopolamine-low-dose API | Scopolamine/Compound of Formula I |
| | | (low dose) |
| 5 | scopolamine-high-dose API | Scopolamine/Compound of Formula I |
| | | (high dose) |
| 6 | healthy-API | Saline/Compound of Formula I |
| | | (high dose) |

Compound of Formula I or its vehicle was administered at 5 ml/kg using p.o. route. The vehicle was 5% DMSO/0.5% methylcellulose in water. Fresh compound solution was prepared daily as following: the required amount of compound will be dissolved in 100% DMSO and then the required amount of 0.5% methylcellulose in water was added. The compounds solubility was checked before the start of the experiment.

On the day of the T-maze, the last treatment was given 1 h before the task.

Donepezil (donepezil hydrochloride) was prepared in saline at a concentration of 0.03 mg/ml and was given p.o. at a dosage volume of 10 ml/kg 1 h before the T-maze trial start. This resulted in dose of 0.3 mg/kg donepezil.

Scopolamine was prepared in saline (vehicle) at a concentration of 0.089 mg/ml (free base) and was given i.p. at a dosage volume of 10 ml/kg 30 min before the T-maze trial start. It resulted in dose of 0.89 mg/kg scopolamine.

### T-CAT Procedure:

The T-maze consists of 2 choice arms and 1 start arm mounted to a square centre. Sliding doors are provided to close specific arms during the force choice alternation task.

During the trials, animal handling and the visibility of the operator were minimized as much as possible.

The experimental protocol consists of one single session, which starts with 1 “forced-choice” trial, followed by 14 “free-choice” trials. In the first “forced-choice” trial, the animal was confined for 5 s in the start arm and then was released while either the left or right goal arm was blocked by closing the sliding door. The animal then negotiated the maze, and eventually entered the open goal arm, and returned to the start position.

Immediately after the return of the animal to the start position, the left or right goal door was opened, and the animal was allowed to choose freely between the left and right goal arm (“free choice trials).

The animal was considered to have entered an arm when it placed its four paws in the arm.

A session was terminated, and the animal was removed from the maze as soon as 14 free-choice trials had been performed or 10 min have elapsed, whatever event occurred first.

The apparatus was cleaned between each animal using alcohol (70%).

The percentage of spontaneous alternations were calculated as number of spontaneous alternations divided by 14 possible free choice.

### Data Analysis:

Statistical analysis was assessed using Anova followed by Fischer's Protected Least Significant Difference for pairwise comparison. p level <0.05 was deemed significant.

### Results

Under control conditions, mice were shown to have an average spontaneous alternation rate of 72% (healthy control group). Administration of scopolamine induced acute cognitive impairment, such that mice treated with 0.89 mg/kg of scopolamine 30 minutes prior to the T-maze alternation task (scopolamine-only group) were expected to have a significantly lower alternation rate of 42% relative to the healthy control group. This is indicative of cognitive impairment.

This cognitive impairment can be largely reversed through the acute pre-treatment of the drug donepezil. Scopolamine-treated mice that also received 0.3 mg/kg of donepezil 1 hour prior to the T-maze alternation task (positive control group) were demonstrated to have a significantly higher spontaneous alternation rate of 65%, compared to the scopolamine-only group.

Chronic pre-treatment of the compound of Formula I was demonstrated to counter the effect of the scopolamine-induced cognitive deficits.

Scopolamine-treated mice receiving chronic pre-treatment of both low-dose and high-dose compound of Formula I were shown to have a significantly higher average alternation rate than the scopolamine-only group of 56%.

The chronic administration of compound of Formula I to mice not treated with scopolamine produced a 64% average spontaneous alternation rate. Demonstrating that the compound of Formula I did not cause any detrimental effects.

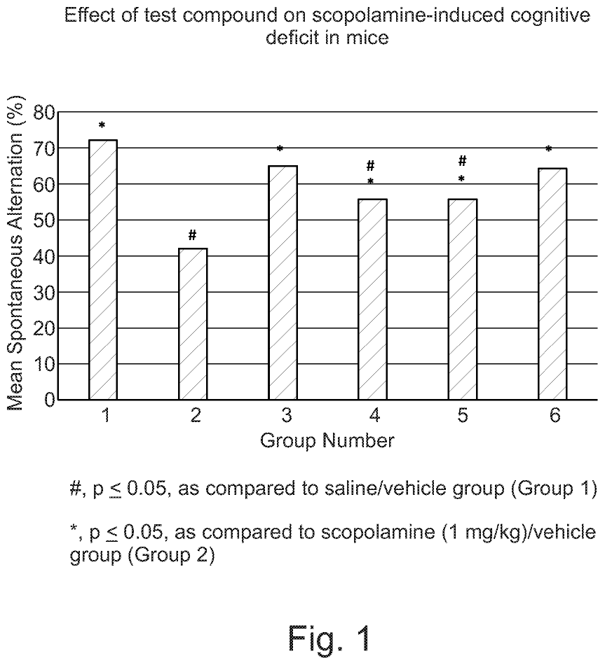

Table 1 below and FIG. 1 describes the results of the spontaneous alternation in the different treatment groups.

TABLE 1
Spontaneous alternation shown by mice in the T-maze

| | | | Spontaneous |
|---|---|---|---|
| Gr. # | Group label | Treatment description | alternation (%) |
| 1 | healthy control | Saline/Vehicle | 72.1 |
| 2 | scopolamine-only | Scopolamine/Vehicle | 42.1 |
| 3 | positive control | Scopolamine/Donepezil | 65.0 |
| | | (0.3 mg/kg) | |
| 4 | scopolamine- | Scopolamine/Compound of | 55.7 |
| | low-dose API | Formula I (5 mg/kg) | |
| 5 | scopolamine- | Scopolamine/Compound of | 55.7 |
| | high-dose API | Formula I (10 mg/kg) | |
| 6 | healthy-API | Saline/Compound of | 64.3 |
| | | Formula I (10 mg/kg) | |

### Conclusion

Treatment with scopolamine significantly reduced the number of spontaneous alternations mice took in the T-maze compared with the healthy control, demonstrating a decline in cognitive function.

This decline in cognitive function was ameliorated by treatment with donepezil in a statistically significant manner.

Mice treated with the compound of Formula I for 30 days prior to the scopolamine induced cognitive deficit were shown to have an increased percentage of spontaneous alternations in comparison to the those treated with vehicle in a statistically significant manner. Such data is indicative that the compound of Formula I was able to improve cognitive dysfunction.

Of interest both the low and high dose treatments of the compound of Formula I were found to result in the same level of increase in spontaneous alternations. This effect is consistent with data reported by Chen et al., 2021, whereby a low dose of CF₃CN was already showing a saturation effect in an animal model of Alzheimer's disease.

Furthermore, the healthy animals treated with compound of Formula I, were shown to have a similar rate of spontaneous alternations compared to healthy vehicle treated animals. Such data is indicative that the compound of Formula I did not impair cognition in healthy animals.

### Example 2: Effect of Test Compound on Sub-Chronic PCP-Induced Deficits in Recognition Memory Using the Novel Object Recognition (Nor) Paradigm in Rats

The novel object recognition test (NOR) test is a two-trial cognitive paradigm that assesses visual recognition memory. A recognition memory task allows the comparison between presented stimuli and previously stored information. Recognition memory is disturbed in a range of human disorders and NOR is widely used in rodents for investigating deficits in a variety of animal models of human conditions where cognition is impaired.

Following initial habituation to the empty test arena, the test consists of two trials. In the first trial, the rats are exposed to two identical objects in an open arena (acquisition phase). In the second trial, following an interval (1 min-6 h, depending on the type of memory and brain region of interest), rats are exposed to two dissimilar objects, one familiar object from the first trial and one novel object (retention phase). Object recognition can be measured as the difference in time spent exploring the familiar and the novel object. Behaviour is recorded and scored using an NOR scoring timer by a trained experimenter who is blind to the treatment groups.

Total object exploration time (defined as the duration of time animals spent licking, sniffing, or touching the object but not including time spent standing or sitting on or leaning against the object) is recorded for each of the familiar and novel objects in the acquisition and retention trials. Discrimination index (defined as the difference in time spent exploring the novel and the familiar objects divided by total time spent exploring both objects) and locomotor activity (defined as movement, measured by the number of lines crossed in both trials) are also calculated. Rats have been shown to spend more time exploring the novel object in the retention phase.

Treatment of the rats with subchronic Phencyclidine (scPCP) induces a selective, long lasting and robust deficit in the NOR paradigm. The present study was conducted to evaluate the ability of the compound of Formula I to reverse scPCP deficit in the NOR paradigm. The effects of scPCP in this paradigm may represent a selective deficit in visual recognition memory which is known to be impaired in schizophrenia and other CNS disorders.

### Materials and Methods

### Drug Preparation and Administration:

Compound of Formula I (5% DMSO in 0.5 Methylcellulose 400 cp) was given at 10 & 30 mg/kg p.o. in a dose volume of 5 ml/kg. This was made fresh daily and constantly stirred prior to dosing. On behavioural days (D1 & D20) Compound of Formula I was administered p.o. 120 mins prior to behavioural testing.

### Animals—Acclimatisation and Housing Conditions:

Rats were housed in groups of 4-5 under standard laboratory conditions, under a 12 hr light: dark cycle (lights on at 07:00 hr).

Testing was carried out in the light phase. Animals had free access to food and water, except during the behavioural testing procedures.

Animals were weighed regularly throughout the experiment.

All procedures were carried out in accordance with the Animal Scientific Procedures Act (UK, 1986) and are approved by the University of Manchester's AWERB (Animal Welfare and Ethical Review Body).

### Novel Object Recognition (NOR) Protocol:

A total of 66 female Lister Hooded rats were used for the NOR study. 56 rats were treated sub-chronically with PCP and 10 with vehicle.

The PCP injection regimen is 2 mg/kg i.p. twice daily for 7 days followed by at least a 7-day drug-free period. This regimen has been found to produce robust and long lasting behavioural and pathological deficits in this paradigm (Neill et al., 2010; Cadinu et al, 2017).

Following the scPCP dosing regimen and prior to behaviour testing, rats were treated as follows:

1. Acute Treatment with Test Compound:

Rats (n=36) were treated acutely with Compound of Formula I (API) at two doses (10 mg/kg (n=18) & 30 mg/kg (n=18), p.o., 120 mins prior to testing on day 1), risperidone (0.1 mg/kg, i.p., 60 min prior to testing) or vehicle (5% DMSO in 0.5 Methylcellulose 400 cp, p.o., 120 mins prior to testing on day 1). Brains (n=3) from each of the API groups were taken immediately following behavioural testing for PK analysis.

2. 20-Day Treatment with Test Compound:

Rats (n=30) were dosed for 20-days with Compound of Formula I (API) at two doses (10 mg/kg (n=15) & 30 mg/kg (n=15), p.o., 120 mins prior to testing on day 20), risperidone (0.1 mg/kg, i.p., 60 min prior to testing) or vehicle (5% DMSO in 0.5 Methylcellulose 400 cp, p.o., 120 mins prior to testing on day 20). Brains (n=3) from each of the API groups were taken immediately following behavioural testing for PK analysis.

### Statistical Analysis:

All data are expressed as mean±s.e.m. (standard error of mean). Exploration time data from NOR in the acquisition and retention phases were analysed separately via repeated measures GLM with factors of drug and exploration time of the 2 objects (2 identical objects in the acquisition phase, and novel and familiar objects in the retention phase). Discrimination index (DI) and locomotor activity data (total number of line crossings in both phases) were analysed via one-way ANOVA. Post-hoc analysis was conducted following a significant one-way ANOVA by the least significant difference (LSD) test (for locomotor activity and DI scores).

Results—Acute Treatment with Test Compound:

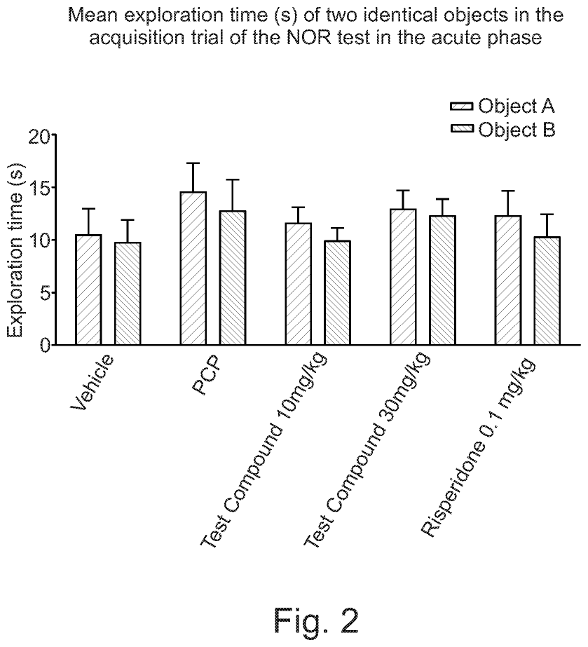

The effect of acute treatment with Compound of Formula I (API) at doses of 10 & 30 mg/kg, p.o. and the positive control risperidone (0.1 mg/kg, i.p.) on the total object exploration time(s) in the acquisition and retention trial are described in Table 2 below and FIGS. 2 to 5.

TABLE 2
Total time spent by rats during the two parts of the NOR trial

| | Acquisition | Retention |
|---|---|---|
| | total exploration | total exploration |
| Treatment | time (s) | time (s) |
| Vehicle + Vehicle | 20.46 ± 3.79 | 23.05 ± 3.08 |
| scPCP + Vehicle | 27.61 ± 5.56 | 27.97 ± 3.18 |
| scPCP + API 10 mg/kg | 21.75 ± 2.46 | 22.55 ± 1.60 |
| scPCP + API 30 mg/kg | 25.49 ± 3.04 | 26.18 ± 2.11 |
| Risperidone 0.1 mg/kg | 22.81 ± 4.03 | 23.10 ± 2.92 |

As can be seen there was no difference in the amount of time spent exploring two identical objects in the acquisition part of the trial (FIG. 2) by any of the treatment groups as was expected.

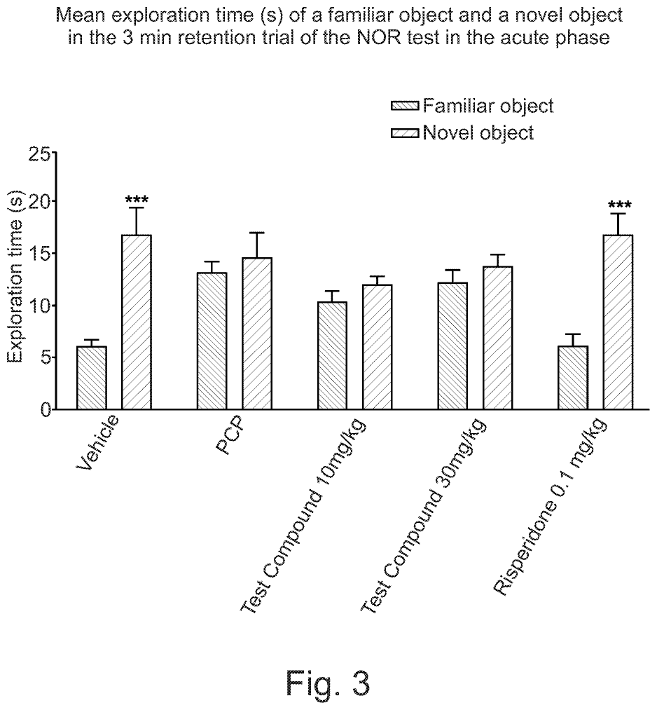

The positive control and the vehicle treated animals showed a statistically significant increase in the amount of time spent exploring the novel object in the retention part of the trial (FIG. 3). The animals not treated (PCP only) and the animals treated acutely with the compound of Formula I were not shown to have a difference in the amount of time exploring the novel or familiar object.

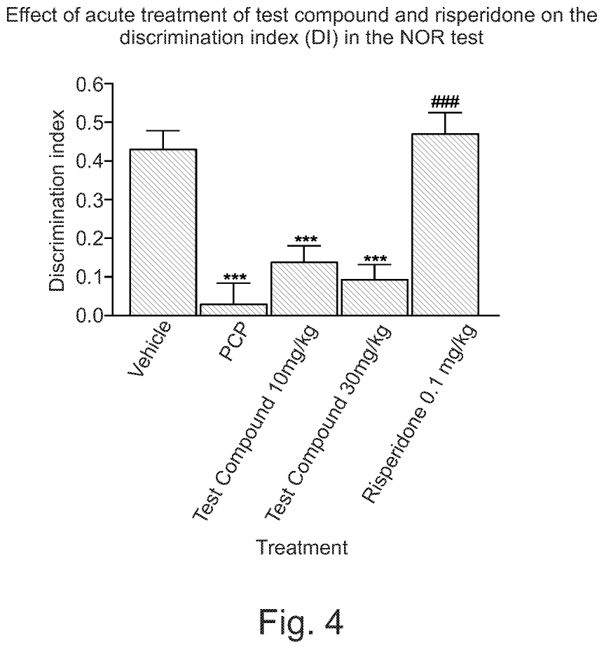

As would be expected from the results shown in FIG. 3, both the vehicle and risperidone treated animals had a higher discrimination index (DI) than those without treatment and those treated with test compound, (FIG. 4).

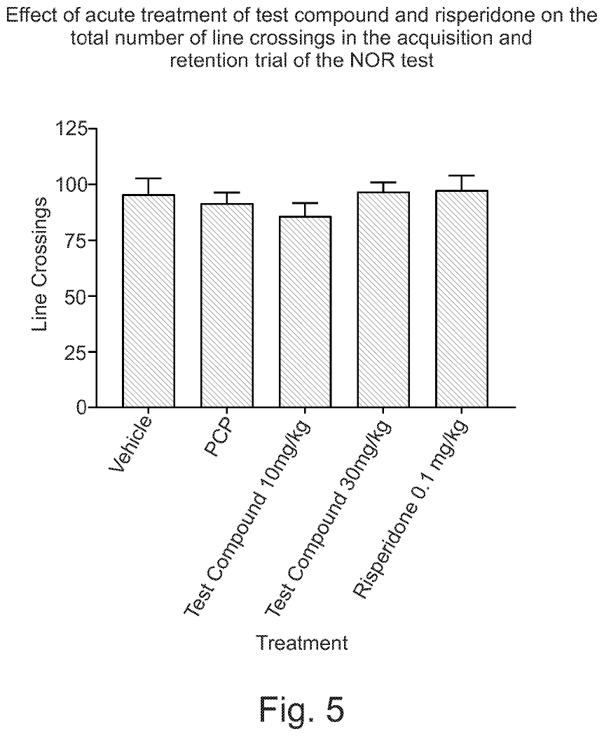

FIG. 5 demonstrates that there was no impact on any of the treatments on locomotor activity as there was no difference in the number of line crossings made within the treatment groups.

Results—20-Day Treatment with Test Compound:

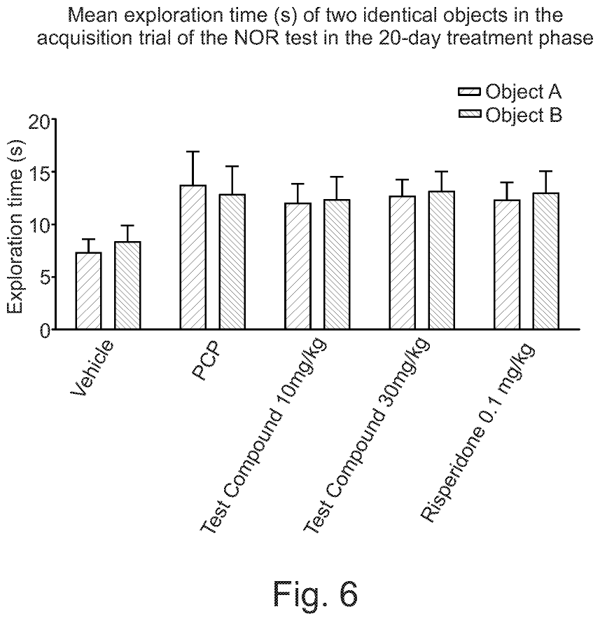

The effect of once-daily treatment with Compound of Formula I (API) at doses of 10 & 30 mg/kg, p.o. for 20 days and acute treatment with the positive control risperidone (0.1 mg/kg, i.p.) on the total object exploration time(s) in the acquisition and retention trial are described in Table 3 below and FIGS. 6 to 9.

TABLE 3
Total time spent by rats during the two parts of the NOR trial

| | Acquisition | Retention |
|---|---|---|
| | total exploration | total exploration |
| Treatment | time (s) | time (s) |
| Vehicle + Vehicle | 15.9 ± 2.6 | 16.0 ± 1.7 |
| scPCP + Vehicle | 26.8 ± 5.3 | 16.1 ± 1.2 |
| scPCP + OT-003 10 mg/kg | 24.6 ± 3.4 | 19.0 ± 1.4 |
| scPCP + OT-003 30 mg/kg | 26.1 ± 3.1 | 20.8 ± 1.2 * |
| Risperidone 0.1 mg/kg | 25.5 ± 3.4 | 17.8 ± 1.1 |

* P < 0.0.5; significant increase in exploration compared to scPCP.

As can be seen there was no difference in the amount of time spent exploring two identical objects in the acquisition part of the trial (FIG. 6) by any of the treatment groups as was expected.

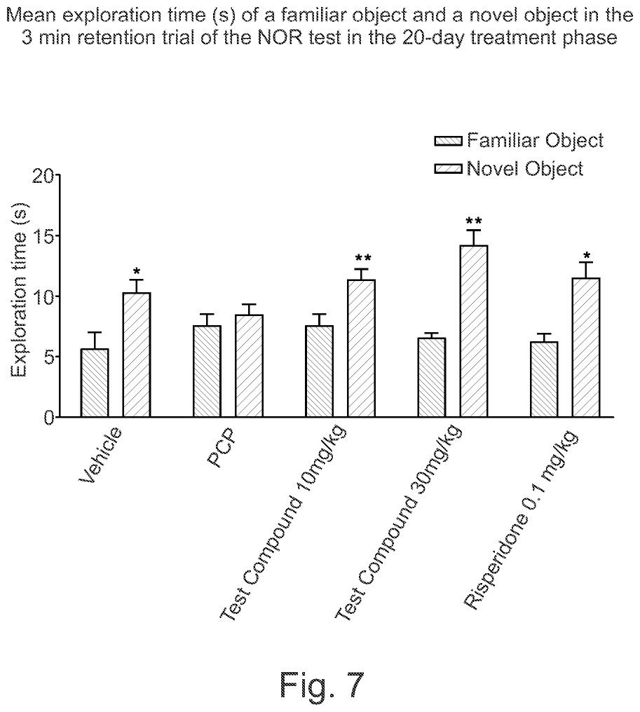

The positive control and the vehicle treated animals showed a statistically significant increase (P<0.05) in the amount of time spent exploring the novel object in the retention part of the trial (FIG. 7). The animals not treated (PCP only) were not shown to have a difference in the amount of time exploring the novel or familiar object.

FIG. 7 further demonstrates a dose dependent statistically significant effect in the amount of time spent exploring the familiar and novel object in the animals treated with the compound of Formula I (** P<0.01). This effect was greater than that seen with the positive control risperidone.

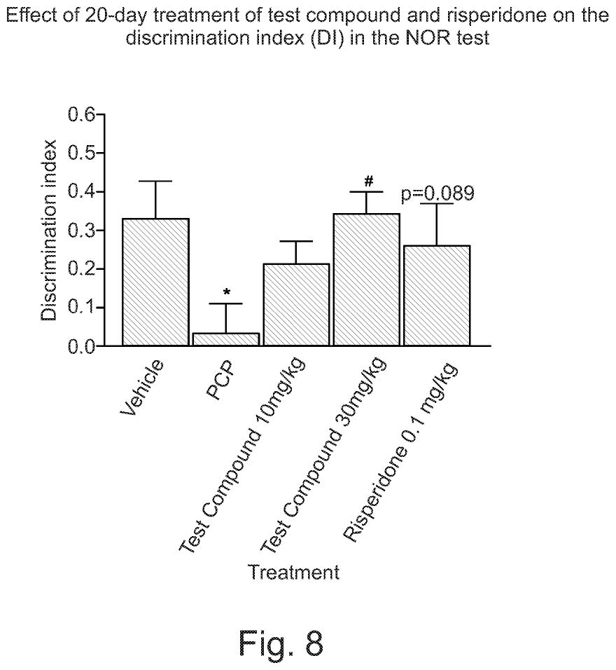

FIG. 8 details the discrimination index (DI) for all the treatment groups. It can be seen that the effect of 20-day treatment of the compound of Formula I at 30 mg/kg produced a statistically significant increase in the DI (P<0.015) compared to the animals without treatment.

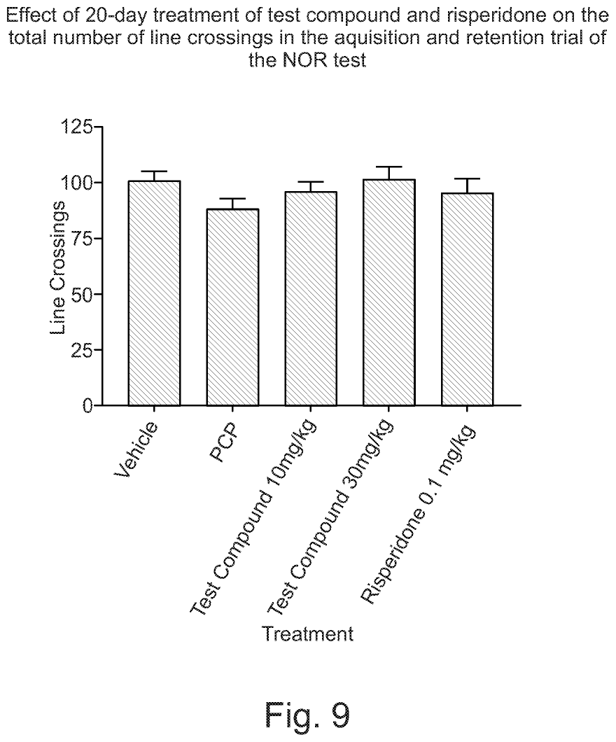

FIG. 9 demonstrates that there was no impact on any of the treatments on locomotor activity as there was no difference in the number of line crossings made within the treatment groups.

### Conclusion

The NOR test has been previously demonstrated to identify compounds that may be useful in the treatment of neurocognitive deficits. The data presented in Example 2 demonstrates that the positive control risperidone, dosed acutely, was able to rescue the deficits induced by scPCP in the rat, indicative that the test was robust.

Importantly, the data additionally demonstrates that dosing animals for a 20-day period with the compound of Formula I is able to rescue the scPCP in a dose dependent manner.

Such a finding provides evidence that the compound of Formula I might be suitable for use in the treatment neurodegenerative diseases or conditions.

1. A compound of Formula I for use in the treatment of neurodegenerative diseases or conditions.

2. A compound of Formula I for use according to claim 1, wherein the neurodegenerative diseases or conditions is selected from the group: Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction; normal pressure hydrocephalus; Parkinson's disease-related cognitive dysfunction; posterior cortical atrophy; primary progressive aphasia; prion disease; progressive supranuclear palsy; Rett syndrome; schizophrenia; stroke-related cognitive dysfunction; traumatic brain injury; traumatic spinal cord injury and vascular dementia.

3. A compound of Formula I for use according to claim 2, wherein the neurodegenerative diseases or conditions is treatment resistant.

4. A compound of Formula I for use according to claim 1, wherein the compound of Formula I is administered with one or more pharmaceutically acceptable excipients.

5. A compound of Formula I for use according to claim 1, wherein the compound of Formula I is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

6. A compound of Formula I for use according to claim 1, wherein the compound of Formula I is administered as a single daily dose.

7. A compound of Formula I for use according to claim 1, wherein the compound of Formula I is administered as multiple daily doses.

8. A compound of Formula I for use according to claim 7, wherein the compound of Formula I is administered two, three, four or five times per day.

9. A compound of Formula I for use according to claims 6 to 8, wherein each dose comprises at least 0.001 mg of the compound of Formula I.

10. A compound of Formula I for use according to claims 6 to 8, wherein each dose comprises between about 0.001 mg and about 500 mg of the compound of Formula I.

11. A compound of Formula I for use according to claims 6 to 8, wherein each dose comprises between about 500 mg and about 1000 mg of the compound of Formula I.

12. The compound of Formula I for use according to claim 1, wherein the compound of Formula I is administered with one or more additional drug products.

13. A method of treating neurodegenerative diseases or conditions in a subject in need thereof, the method comprising administering to the subject a therapeutically effective amount of the compound of Formula I.
